(19) United States
(12) Patent Application Publication
(10) Pub. No.: US 2014/0142676 A1
(43) Pub. Date: May 22, 2014

(54) ELECTRODE-RETAINING HEADBAND

(71) Applicant: NORDOCS TECHNOLOGIES INC., Ottawa (CA)

(72) Inventors: Paul Gardin, Ottawa (CA); Daniel Shapiro, Ottawa (CA); Patrick D.H. Williams, Chelsea (CA); Kevin J. Bailey, Ottawa (CA); Mathieu Lemay, Ottawa (CA); Shazib Shaukat, Ottawa (CA); Stanley Shapiro, Sudbury (CA); Brian Dressler, Sudbury (CA)

(73) Assignee: NorDocs Technologies Inc., Ottawa (CA)

(21) Appl. No.: 13/679,823

(22) Filed: Nov. 16, 2012

(51) Int. Cl. A61N 1/04

(52) U.S. Cl. CPC A61N 1/0404
USPC 607/139

(57) ABSTRACT

A headband for retaining electrodes in position on a patient's head is disclosed, having an adjustable frame, straps for holding electrodes against the head, each strap fastened to the headband by an adjustable support, and the supports having weights removably coupled thereto such that gravity pulls the headband down against the electrodes. The headband may be used for transcranial electrical stimulation treatment or other treatment by electrodes. Further disclosed is a method of retaining electrodes on a patient's head, having the steps of positioning a headband having straps on the head, tightening the headband and straps, inserting electrodes between the straps of the headband and the head, mounting weights to bias the headband downwardly, and loosening the straps for greater comfort. The headband and straps may have markings to facilitate placement according to an electrode marking system.

### BACKGROUND OF THE INVENTION

1. Field of the Invention

The invention relates to a harness for the head, and in particular to a headband for retaining electrodes in position on a head.

2. Description of the Prior Art

The prevalence of Transcranial Stimulation devices and techniques has created a need for precise electrode placement on the head, wherein the electrodes may be positioned in a particular manner to target particular parts of the brain. The traditional solution to positioning electrodes and saline-soaked sponges uses an elastic bandage, which is wrapped around the head and over the positioned electrodes. The elastic bandage is susceptible to upward creep, as is it wrapped around a tapering part of the head and movement of the head results in movement, or creep, of the bandage up and off the head, due to the contracting force of the contracting elastic bandage. The elastic bandage may also absorb liquid, which can result in a lessening of the definition of the electrical contact patch of the electrode, as the saline solution used in the sponge to transmit current from the electrode into the skin absorbs into the bandage and conducts current through the bandage. The saline leak shunts the current from the intended electrode positioning to a position adjacent, inadvertently stimulating part of the brain that is not intended to receive stimulation, creating a short-circuit with the other electrode, or changing the density and distribution of current. The application of electrodes and an elastic bandage is very difficult by one person as the electrode sponges must be held in position while the elastic bandage is wrapped around the head, and is normally performed with two people, the first holding the electrodes and the second wrapping the bandage. This results in a time-consuming, awkward and inefficient process. Variation on this approach is a rubber strap with perforations and a snap-type, button or buckle closure, or a non-elastic belt with a clasp or buckle. These variations have similar drawbacks such as difficult application and susceptibility to upward creep.

An alternative in the prior art is a balaclava-type electrode retainer, a stretchy balaclava with a larger face hole, which fits over the head and has several holes in the top of the balaclava to fit sponges into, for connection to the electrodes. Although this counteracts the forces pulling the elastic bandage described above up and off the head by having a chin-holding component, the elastic material of the balaclava can shift during treatment unintentionally changing the placement of the electrodes and sponges. Holes are made in the balaclava to permit placement of sponges, however these holes are permanent and their location cannot be altered. The application of the sponges within the balaclava again requires two people, since the wearer of the balaclava cannot see the top so as to position sponges within the holes. Furthermore, any absorbent material in the balaclava may absorb the saline solution and unintentionally shunt the current through a different portion of the brain than targeted, or short-circuit one electrode to the other. The balaclava may also be too warm and uncomfortable to wear in a clinic environment, as balaclavas are traditionally intended to keep head and neck warm during cold weather.

Retaining the sponge to the head poses a particular set of problems. For optimal placement and contact, the sponge should follow the curvature of the head and to do this pressure must be applied across the area of the sponge. Sponges are not generally manufactured in a curved shape, rather as they are flexible they are manufactured in a planar shape and pushed to conform with the surface to which they are to be applied. Even if manufactured in a curved shape pressure must still be applied to hold the sponges in contact with the head. If the sponge does not follow the curvature of the head, then the contact area is not precise or as large as it can be. For example, when a strap passes over the sponge to hold it in place, the sponge is pushed against the head where it is present below the strap, but the portions that project from beneath the strap balloon out to either side of the strap and are often not in contact with the head since they don't follow the curvature. Therefore it is only a portion of the sponge surface area that is in firm contact with the head.

Ensuring sufficient contact area between the sponge and the head is important both to ensure effectiveness of the treatment, and also to ensure that the current delivered during the treatment does not burn or otherwise injure the skin of the patient, as decreased contact area corresponds to increased electrical current density, and excessive current density will cause skin burns, pain and irritation. In other fields requiring electrode application to the head—such as electroencephalogram (EEG)—the electrodes do not deliver current to the patient, and thus most pre-existing electrode-head applications do not have the same concern for ensuring contact area to prevent skin burns.

### SUMMARY OF THE PRESENT INVENTION

A headband for retaining electrodes in position on a patient's head is disclosed, the headband comprising an adjustable frame for mounting on the head; one or more straps for retaining electrodes against the head, each strap having a first and second end; a support connected to each end of the straps, each support connected to the frame and having a weight mount; and one or more weights for attachment to the weight mounts to bias the frame downwardly against the head.

A further embodiment is disclosed wherein the support is removably mounted on the frame. An alternate embodiment is disclosed wherein the support length is adjustable. The support may be connected to the frame by an adjustable sleeve. Further, the headband is disclosed wherein the support is releasably connected to the frame by a fastener selected from the group consisting of a snap, a hook and loop fastener, a button and hole, a clasp, and a keyhole fastener.

A further embodiment discloses the strap length is adjustable. Further, a headband wherein the strap is non-elastic, non-absorbent and non-conductive is disclosed. In one embodiment, the one or more straps are shaped for the curvature of the head. Further in an embodiment each weight has a corresponding opposite weight so as to balance the headband on the head. The weight may be wrapped around the head. The straps or headband may be marked with distance markings to assist with positioning electrodes. The straps or headband may be marked according to an electrode placement system to assist with positioning electrodes.

Disclosed is the use of the headband for transcranial electrical stimulation treatment and/or for a transcranial electrical measurement or imaging application.

Further disclosed is a method of retaining electrodes on a patient's head, comprising the steps of: positioning a headband having straps on the head; inserting electrodes between the straps of the headband and the head; and mounting weights to bias the headband downwardly. In one embodiment the method further comprises the step of tightening the straps such that the electrodes conform to the shape of the head. The method may further comprise the step of loosening the headband for increased comfort.

Also disclosed is a method of retaining electrodes on a patient's head, comprising the steps of: locating and marking the desired electrode positions on the head; positioning a headband on the head wherein the headband has straps; positioning the straps such that they pass over top of the marked locations; tightening the headband to conform to the shape of the head; inserting electrodes between the straps of the headband and the head such that the electrodes are centered at the marked locations; tightening the straps such that the electrodes are held firmly to the head; and mounting weights to bias the headband downwardly.

Further disclosed is the method above further comprising the step of referencing marking on the straps and headband to position the electrodes in accordance with an electrode placement system, and the method above further comprising the step of loosening the headband for increased comfort, wherein gravity biases the headband downwardly to hold the electrodes onto the head.

### BRIEF DESCRIPTION OF THE DRAWINGS

FIG. 1 shows an elevation view of the headband in position on a patient's head;

FIG. 2 shows an elevation view of the front of the headband;

FIG. 3 shows an elevation view of the rear of the headband;

FIG. 4 shows an elevation view of the side of the headband;

FIG. 5 shows a plan view from the top of the headband;

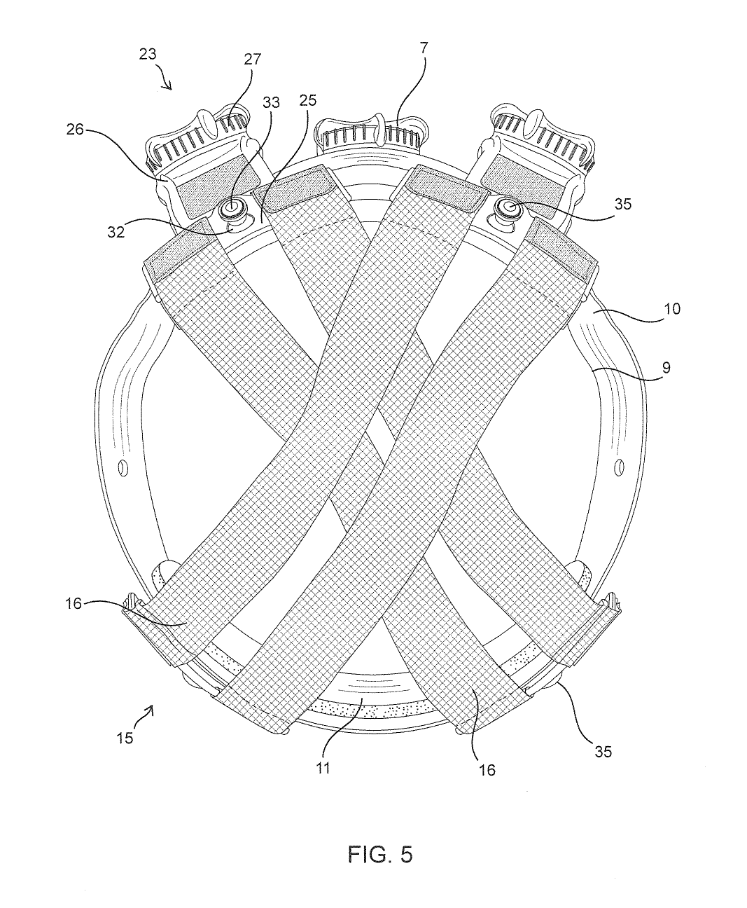

FIG. 6 shows a plan view from below of the headband;

FIG. 7 shows a detail view of an adjustable support; and

FIG. 8 shows an elevation view of the headband in position on a patient's head with a weight coupled thereto.

### DETAILED DESCRIPTION OF THE PRESENT INVENTION

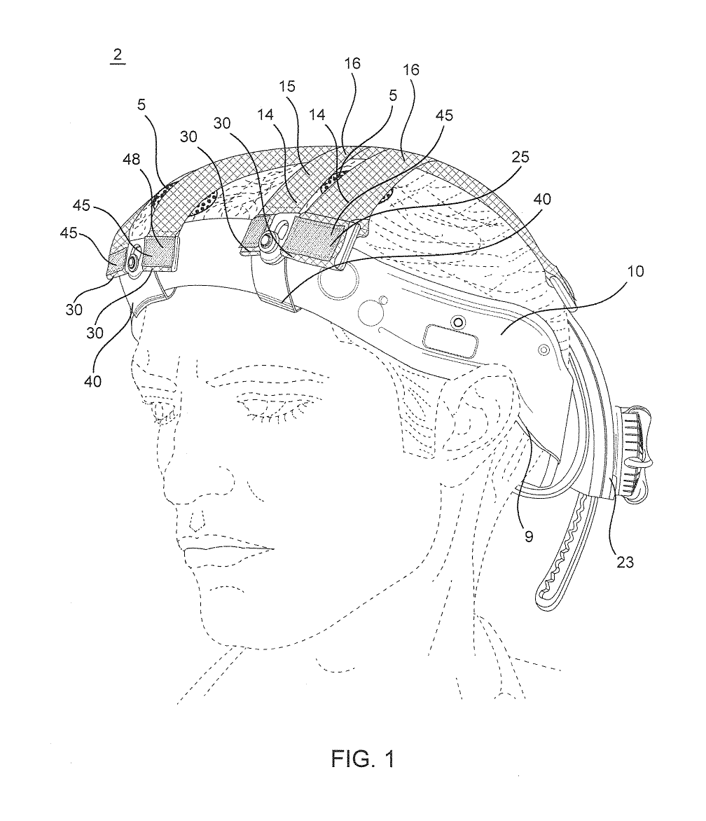

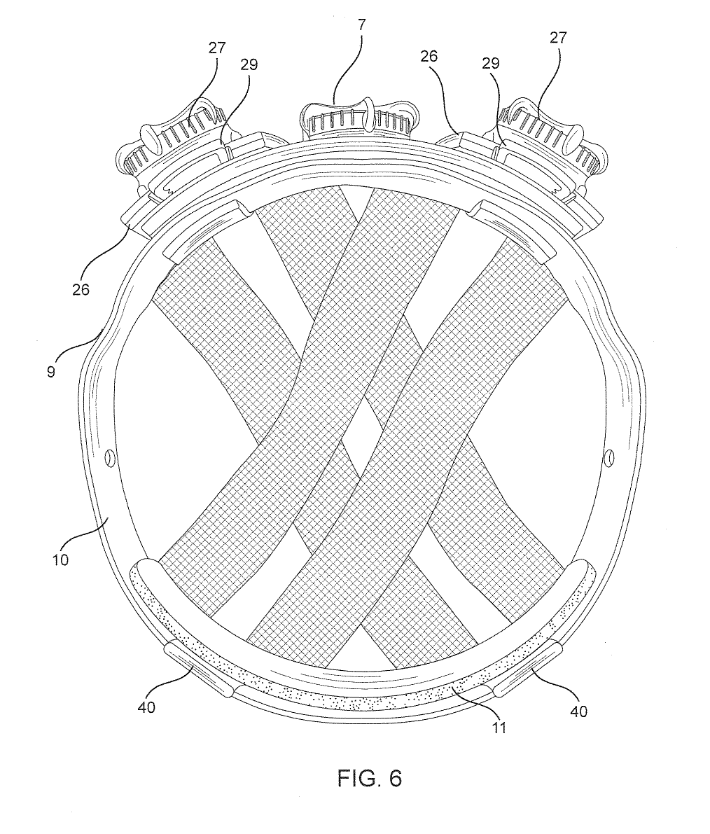

With reference to FIGS. 1, 3, 6 and 7, the electrode-retaining headband 2 consists of a main frame 10, adjustable and for mounting on the head of a patient, having one or more straps 15, each strap composed of one or more strips 16, each strap 15 connected at its ends 14 to opposite sides of the main frame 10 for retaining electrodes 5 against the head (shown in stippled lines). Each of the straps 15 is removably attached to approximately opposite sides of the frame 10 by curved supports 25. The frame fits more or less circumferentially around the head, from forehead to the rear of the cranium and above the ears at the side, and is adjustable for different head sizes by an adjustable mechanism 7 such as a gear mechanism, hook-and-loop fasteners, snaps or ratchets, as are known in the art for hats, hardhats, helmets and caps. The frame 10 may have a cushion 11 therein for contact with the forehead, as well as a rear cushion 12 for contact with the occipital area at the rear of the head. It may also have an ear curvature 9 on each side to accommodate ears.

The strap 15 is formed of one or two strips 16 of non-elastic, flexible, low-friction and hydrophobic or non-absorbent material such as nylon. In another embodiment, the strap 15 is formed of a single, wide strip 16 of material. In either case, the sponges are to be positioned beneath the strap 15 for treatment of a patient. The length of the strap 15 is adjustable by means of adjustability in the strap 15 (not shown) and/or adjustability of the support 25. The non-elastic nature of the material allows it to be firmly positioned across the top of the head, without unintentional movement on the part of the frame 10 due to elasticity. A low coefficient of friction permits the straps to slide by one another and ease the movement of the straps into different positions when two or more straps 15 are used to cross the top of the head and cross each other. Moreover, a low-friction material does not pinch hair or scalp. The strip material should be hydrophobic and non-absorbent to reduce the possibility of a short circuit due to the saline of the sponges being absorbed in the material and inadvertently increasing the sponge area or causing a short-circuit between electrodes.

The strip material should also be a very poor conductor of electricity. In another embodiment, the strap may be made of flexible or rigid molded plastics.

In one embodiment, a support 25 to connect the strap 15 to the frame 10 is fastened to each end of the strap 15, and one or both end-mounted supports 25 are adjustable for length. In a second embodiment, one end of the strap 15 is connected directly to the frame 10 by a pivoting pin or rivet (not shown) for example, and the other end is connected to a support 25, adjustable for length. hi that embodiment, the pin or rivet is the support 25 enabling the pivoting connection. In one embodiment where the straps 15 are connected directly to the frame 10, the straps 15 may be shaped, through sewing or molding, for example, such that it is narrow at each end, but wide in the middle, and the path from connection to connection though any point at the wide center is the same, such that the strap has a spherical shape. As a result, tension across the strap against the head is equal at all points in the wide center, holding the sponge against the head evenly across the strap 15 width. The support 25 has protruding arms 30 or a slot (not shown) therein to receive the strips 16, and the strips 16 may be sewn around the arms 30, or glued thereto, or fastened thereto in any other manner known in the art for fastening a flexible strap to a rigid plastic support. One skilled in the art would appreciate that the support 25 may be made of plastic, nylon, metal, wood or any other rigid or semi-rigid material, and may be curved to accommodate the curvature of a patient's head and apply even pressure. In another embodiment, the strap 15 may be pivotally affixed to the frame 10 at a first end, and removably attached to the frame 10 at a second end, instead of removably attached at both ends. The support 25 may also contain a mechanism for shortening or lengthening the strap, or the strap itself may double back to form a means of shortening or lengthening the strap, as is known in the art. The support 25 is intended to be attached first and shortened or tightened after, so it may be adjusted by one hand. Ideally, the straps 15 are positioned over the apex of the head.

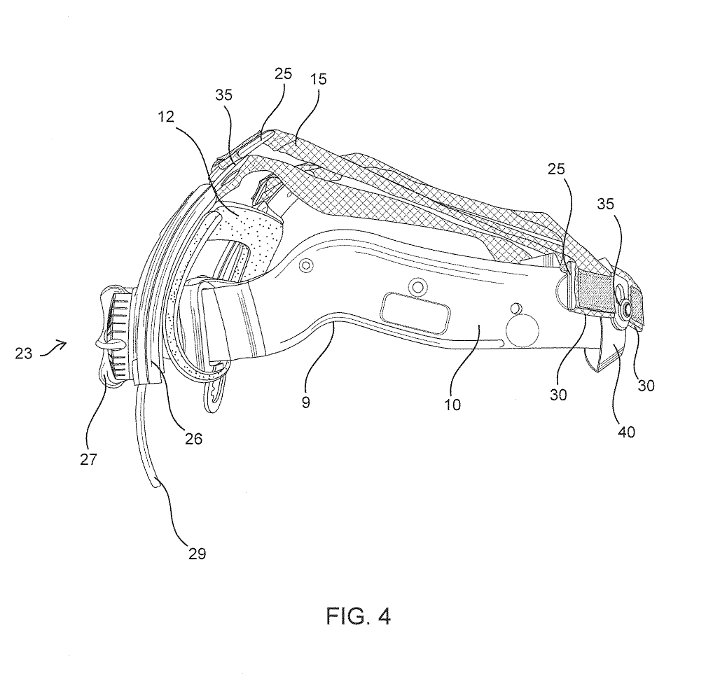

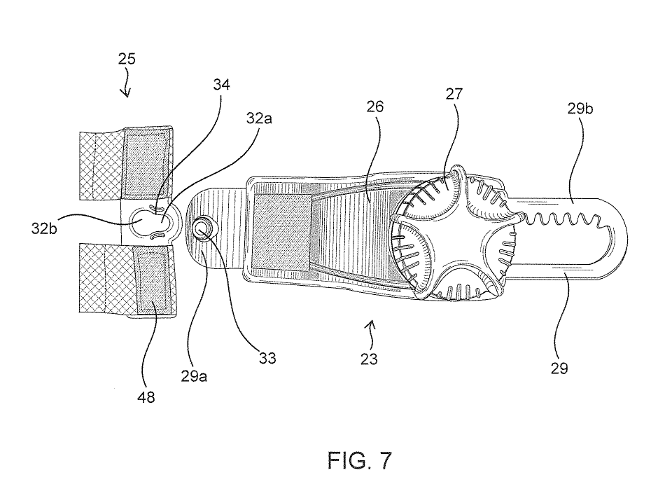

In the preferred embodiment, and with reference to FIGS. 4 and 7, a length-adjustment mechanism 23 is mounted between the support 25 and the frame 10, which uses a ratchet or gear tightening mechanism to tighten and loosen the straps 15. The adjustment mechanism's case 26, which is pivotally affixed to the frame 10, has a dial 27 which moves a gear (not shown) within the case 26 along a set of teeth 28 near one end of an extension 29 that passes through the case 26 and extends from both ends of the case 26, the extension 29 having a support end 29a and a toothed end 29b. The support end 29a of the extension 29 is affixed to the support 25 by a releasable fastening means 35. Turning the dial 27 one way extends the support end of the extension 29 from the case 26, and turning the dial 27 the other way draws the support end of the extension 29 within the case 26. Other embodiments may include straps 15 with multiple fastening points to achieve different lengths; webbing strips terminating each strap end which can be adjusted in length through cinching hardware, like back-pack shoulder straps; chords of plastic, rope or elastic passing through cleats; and cable ratchet systems consisting of a thumb-wheel and a cable allowing the length to be adjusted without free ends of the strap protruding beyond the frame.

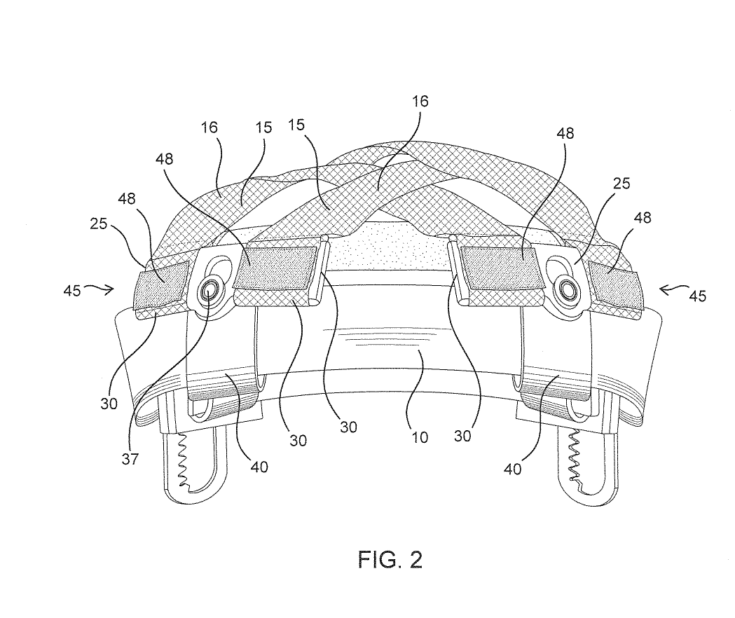

With reference to FIG. 2, the support 25 releasably connects with the frame 10 or the length-adjustment mechanism 23 by a releasable fastening means 35. The fastening means 35 is preferably operable by only one hand so the patient may operate the support fastening him- or herself, or the medical staff may operate two fastening means 35 at one time. In FIG. 7 a keyhole fastener comprising a keyhole 32, pin 33 for positioning therein and a restriction 34 at the midpoint to releasably lock the pin within the narrow portion 32a of the keyhole 32. The pin 33 has a wide head that may pass through the wide portion 32b of the keyhole 32, but not the narrow portion 32a. The resilient restriction 34 makes the entrance to the narrow portion 32a narrower than the diameter of the pin 33, and temporarily deforms under pressure by the pin 33, to allow the pin to enter the narrow portion 32a and be held therein until an opposite application of force pushes the pin 33 through the restriction again. The pin 33 requires the application of force to enter the narrow portion 32a and to be removed therefrom. The fastening means 35 may consist of a snap, a button and hole, a clasp, a keyhole fastener 32, 33 with or without a restriction 34 at an entrance to the narrow portion 32a, or hook-and-loop fasteners. Other fasteners known in the art may be used to releasably connect the support 25 and the frame 10.

The support 25 may be attached directly to the frame 10 by a fastening means 35 such as a keyhole fastener, a socket for a snap or a hole for a button to fit through. On the frame 10 is affixed a corresponding mounting point 37(not shown) to the fastening means 35, for example a pin 33 for the keyhole fastener of the fastening means 35, a snap to fit into a socket, or a button to fit through a hole on the strap. In the embodiment shown in FIG. 7 the corresponding mount point 37 is a pin for a keyhole fastener. In another embodiment, with reference to FIGS. 2 and 7, the support 25 is not attached directly to the frame and instead is attached to a sliding sleeve 40 slidably affixed around the frame and slidably moveable along the frame, wherein the corresponding mount point 37, in this embodiment a pin 33, as described above is positioned on the sliding sleeve 40. These slidable sleeves 40 allow the fastening means 35 of the straps 15 to be moved to any position around the periphery of the head, though the endpoints of a strap 15 will generally be on opposite sides of the head, the strap passing over the apex of the head. In another embodiment, rather than the movable sleeves 40, a series of mount points 37 are positioned around the frame 10, from which different adjustable positions may be chosen.

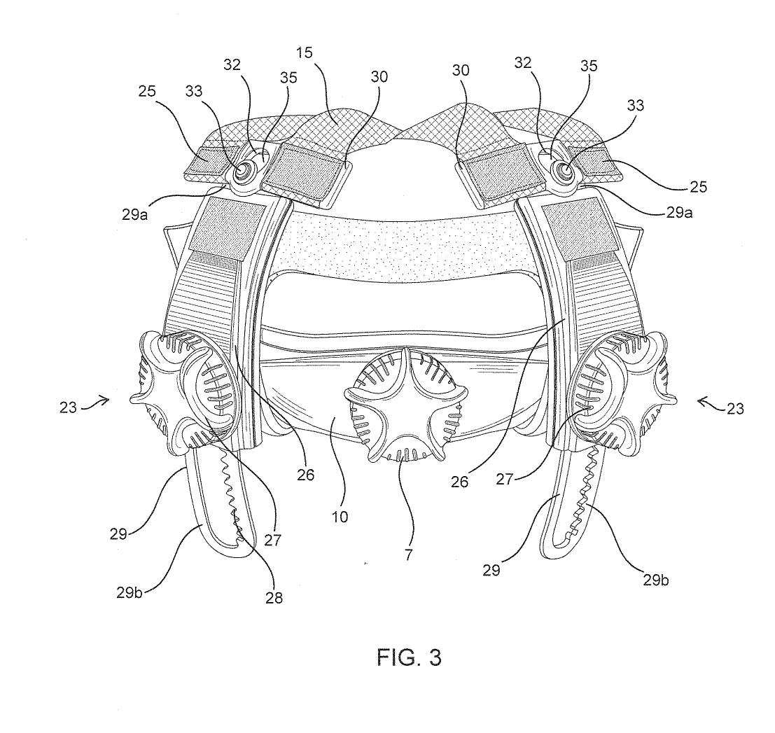

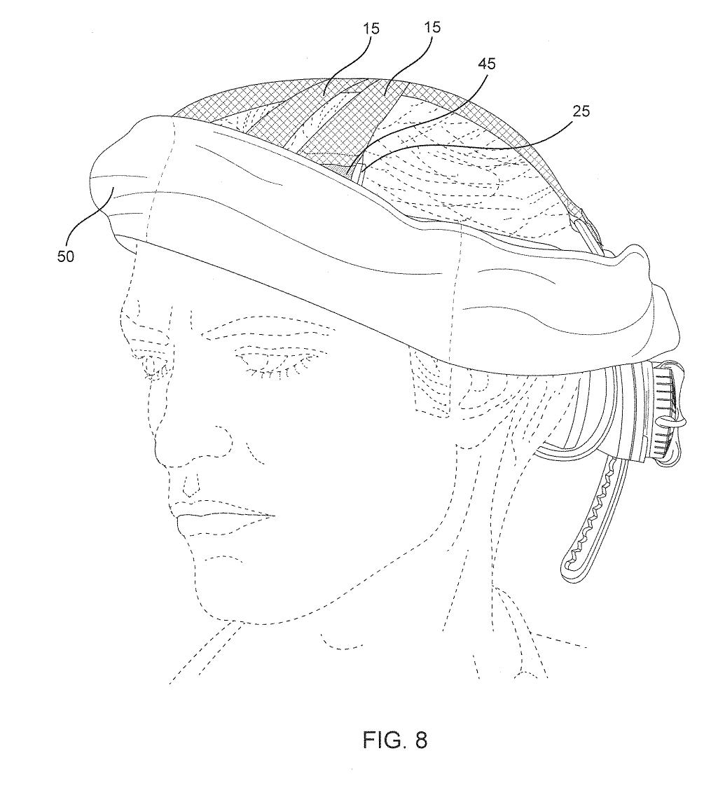

With reference to FIGS. 2 and 3, each support 25 has a weight coupling 45 for holding weights 47 (not shown) thereon. In the preferred embodiment, the weight coupling 45 consists of two pads 48 of hook-and-loop fastening material sewn to each end of the strip 16, to couple with a weight 47 composed of a weighty substance within a closed sack that binds with the hook-and-loop material of the weight coupling 45. The weight coupling 45 may alternatively consist of mounts, fasteners, snaps, zippers, buttons or other mounts or fasteners known in the art. Automatic fasteners like VELCRO™ are preferred. The frame 10 and straps 15 could also include hook elements to catch the weight band. In one embodiment the weight 47 may be around the periphery of a nylon skull-cap sort of hat that pushes directly on the sponges and electrodes rather than pulling on the straps 15. In one embodiment the weights 47 are multiple sacks from a durable material that contain a weighty substance such as sand, gel or a metal such as lead, are closed and have an attachment point 49 thereon, for example a section of hook fabric, a hole for attachment to a button, or a pin, for fastening to the corresponding weight coupling 45. The durable material may be cloth, nylon cloth, rubber, hook-and-loop fabric or any other material that is robust and holds weighty substances without leaking. With reference to FIG. 8 an alternative to the multiple sack arrangement described above is a single flexible tube weight 50 filled with a weighty substance that can be easily draped around the frame and affixes automatically to the supports 25 or to attachment points (not shown) on the frame 10, due to the VELCRO™ loop nature of the tubes material or the geometry of the frame. This automatically balances the weight around the circumference of the head. Further weight couplings 45 may be positioned around the frame 10, for example in an embodiment where a support is present on only one side of the strap 15, in the location where other side of the strap 15 is connected to the frame 10, further weight couplings 45 may be present. Ideally, weights will be distributed around the frame 10 to balance the frame 10 on the head, that is, each weight 47 is counterbalanced by another weight 47 positioned opposite.

In order to apply the headband 2, the electrode positions must be determined, according to the treatment that is to be given. In one embodiment, the straps 15 and/or the frame 10 are printed with measurements or other alignment marks to facilitate determining the correct electrode positions. In one embodiment, the straps 15 and/or frame 10 are printed with marks corresponding to an electrode placement system. An electrode placement system may be any standardized system of locating a consistent area of the scalp. A preferred electrode placement system is the International 10-20 system—a well-known method of applying scalp electrodes based on the percentage distance between anatomical landmarks. In the International 10-20 system each site has a letter to identify the lobe and a number to identify the hemisphere location. The letters F, T, C, P and O stand for frontal, temporal, central, parietal, and occipital lobes, respectively. The “C” letter is only used for identification purposes only as there is no central lobe. A “z” (zero) refers to an electrode placed on the midline. Even numbers (2,4,6,8) refer to electrode positions on the right hemisphere, whereas odd numbers (1,3,5,7) refer to those on the left hemisphere. Therefore the markings on the headband or straps would coincide with the likely location of these points, for example “P3” may be marked a third of the way from the rear along the strap for the left parietal lobe, “F7” may be marked near the front support of the strap 15 for the left frontal lobe, and “T4” may be marked on the frame 10 above the ear for the right temporal lobe. Other potential electrode placement systems may include the International 10-10 system or the Modified Combinatorial Nomenclature, which also rely on distances between anatomical landmarks, and these systems may be marked on the frame 10 and straps 15 accordingly. Alternatively, the frame 10 and straps 15 may be marked with distance markings in metric or imperial units to determine positioning of the electrodes.

The headband is then placed over the head so the frame 10 is positioned across the forehead and around the back of the head of the patient. Just before the frame 10 is on the head and the straps 15 are tightened to fit the head snugly, the electrodes may be adjusted beneath the straps 15 to sit the appropriate positions for the proper treatment. The pads are beneath the straps and are typically not positioned beneath the frame 10, rather the frame 10 is for aligning the straps on the head. Once the headband 2 is snug and the electrodes 17 are in position, the weights 47 are applied to bias the frame 10 and one or more straps 15 down against the head, preferably in a counterbalanced mode such that the frame is balanced across the head and not biased to one side. Preferably the weights counterbalance due to the way they are manufactured as a single unit that wraps around the head in a way that is not dependant on head size. Once the weights 47 are positioned on the frame 10, the frame 10 may be loosened as the gravity on the weights 47, and not the tightness of the frame 10, holds the electrodes in place on the patient's head. Holding the weights from the frame 10 in this fashion, using the pull of gravity to hold the electrodes in place, is much more comfortable than the frictional force from having the frame 10 and straps 15 tightened for the duration of the treatment to hold the electrodes. Having the weight on the head, assisted in its positioning by the loosened frame 10 and straps 15, is also more comfortable than a chin strap. The amount of weight required to provide adequate downward force is low enough that the wide majority of patients, including children, do not have trouble supporting the system on their head for the duration of a treatment.

For ease of maintenance, the materials of the electrode-retaining headband 2 are easily washable, being made of a plastic that is alcohol-resistant. The parts of the headband are smoothed, on the surfaces that touch the patient's head to reduce pressure spots. In one embodiment, the headband parts are made of bacteria-resistant plastic. Any soft materials that are used to cushion the headband may be removable and washable or replaceable to maintain a hygienic condition for use by multiple patients.

Many modifications and other embodiments of the invention will come to the mind of a person skilled in the art having the benefit of the teachings presented in the foregoing description and associated drawings. Therefore, it is understood that the invention is not to be limited to the specific embodiment disclosed, and that modifications and embodiments are intended to be included within the scope of the appended claims.

We claim:

1. A headband for retaining electrodes in position on a patient's head, the headband comprising:
a. an adjustable frame for mounting on the head;
b. one or more straps for retaining electrodes against the head, each strap having a first and second end;
c. a support connected to each end of the straps, each support connected to the frame and having a weight mount; and
d. one or more weights for attachment to the weight mounts to bias the frame downwardly against the head.

2. The headband according to claim 1, wherein the support is removably mounted on the frame.

3. The headband according to claim 1, wherein the support length is adjustable.

4. The headband according to claim 1, wherein the support is connected to the frame by an adjustable sleeve.

5. The headband according to claim 1, wherein the support is releasably connected to the frame by a fastener selected from the group consisting of a snap, a hook and loop fastener, a button and hole, a clasp, and a keyhole fastener.

6. The headband according to claim 1, wherein the strap length is adjustable.

7. The headband according to claim 1, wherein the strap is non-elastic, non-absorbent and non-conductive.

8. The headband according to claim 1, wherein the one or more straps are shaped for the curvature of the head.

9. The headband according to claim 1, wherein each weight has a corresponding opposite weight so as to balance the headband on the head.

10. The headband according to claim 1, wherein the weight is wrapped around the head.

11. The headband according to claim 1, wherein the straps or headband are marked with distance markings to assist with positioning electrodes.

12. The headband according to claim 1, wherein the straps or headband are marked according to an electrode placement system to assist with positioning electrodes.

13. The use of the headband according to claim 1 for transcranial electrical stimulation treatment.

14. The use of the headband according to claim 1 for a transcranial electrical measurement or imaging application.

15. A method of retaining electrodes on a patient's head, comprising the steps of:
a. positioning a headband having straps on the head;
b. inserting electrodes between the straps of the headband and the head; and
c. mounting weights to bias the headband downwardly.

16. The method according to claim 15, further comprising the step of tightening the straps such that the electrodes conform to the shape of the head.

17. The method according to claim 15, further comprising the step of loosening the headband for increased comfort.

18. A method of retaining electrodes on a patient's head, comprising the steps of:
a. locating and marking the desired electrode positions on the head;
b. positioning a headband on the head wherein the headband has straps;
c. positioning the straps such that they pass over top of the marked locations;
d. tightening the headband to conform to the shape of the head;
e. inserting electrodes between the straps of the headband and the head such that the electrodes are centered at the marked locations;
f. tightening the straps such that the electrodes are held firmly to the head; and
g. mounting weights to bias the headband downwardly.

19. The method according to claim 18 further comprising the step of referencing markings on the straps and headband to position the electrodes in accordance with an electrode placement system.

20. The method according to claim 18, further comprising the step of loosening the headband for increased comfort, wherein gravity biases the headband downwardly to hold the electrodes onto the head.
